(19) United States
(12) Patent Application Publication
(10) Pub. No.: US 2005/0036944 A1
(43) Pub. Date: Feb. 17, 2005

(54) Diffusion-weighted parallel imaging with navigator-signal-based phase correction

(76) Inventors: Johan Samuel Van Den Brink, Eindhoven (NL); Miha Fuderer, Eindhoven (NL)

Correspondence Address: Thomas M Lundin;Philips Intellectual Property & Standards, 595 Miner Road, Cleveland, OH, 44143, US

(21) Appl. No.: 10/498,634

(22) PCT Filed: Dec. 2, 2002
(86) PCT No.: PCT/IB02/05113

(30) Foreign Application Priority Data
Dec. 14, 2001 (EP) 0120909.4

(51) Int. Cl. A61K 49/00; A61B 5/055

(52) U.S. Cl. 424/9.3; 600/410

(57) ABSTRACT

A magnetic resonance imaging method for forming an image of an object from a plurality of signals acquired by an array of multiple receiver antennae, wherein spins are excitated in a part of the object. MR signals are measured along a predetermined trajectory containing a plurality of lines in k-space by application of a read gradient and other gradients. Further, a navigator gradient is applied for the measurement of navigator MR signals and an additional gradient is applied in order to achieve diffusion sensitivity of the MR signal, wherein phase corrections are determined from phases and moduli of the navigator MR signals so as to correct the measured MR signals. An image of the part of the object is determined from the corrected MR signals. The corrected phase is determined from the weighted phase difference between a reference navigator signal for each antenna and the actual navigator MR signal of said antenna.

The invention relates to a magnetic resonance (MR) method for the imaging of an object arranged in a steady magnetic field, whereas the following steps being repeatedly executed according to said method:
excitation of spins in a part of the object,
measurement of MR signals along a predetermined trajectory containing a plurality of lines in k-space by application of a read gradient and other gradients,
application of a navigator gradient for the measurement of navigator MR-signals,
said method also including the determination of a phase correction from phases and moduli of the measured navigator MR signals so as to correct the measured MR signals and the determination of an image of the part of the object from the corrected MR signals.

The invention also relates to an MR device for carrying out such a method.

A method of the kind set forth is known from WO-A-98/47015, in which the method is applied to the specific case of diffusion weighted imaging. Here, a corrected phase is determined for a measured navigator MR signal from a measuring point, for which the modulus of the measured navigator MR signal is smaller than a threshold value, from the phases of the measured navigator MR signal form different reference measuring points for which the moduli of the navigator MR signal exceed the threshold value. The method is based on the fact that the presence of a strong diffusion motion due to a high value of the additional gradient reduces the value of the moduli of the measuring points in the navigator MR signal which correspond to regions of the brain which contain a large quantity of cerebrospinal fluid (CSF). Because of the low value of the moduli, the error in the determination of the phase increases. With the described method the artefacts in the MR image can be reduced when for the measuring points having a modulus smaller than the threshold value the corrected phase is determined from the phases of the various reference measuring points of the navigator MR signal for which the phase can be determined with a sufficiently small error.

In magnetic resonance imaging there is a general tendency to obtain acceptable images within shorter periods of time. For this reason the sensitivity encoding method called “SENSE” has recently been developed by the Institute of Biomedical Engineering and Medical Informations, University and ETH Zürich, Switzerland. The SENSE method is based on an algorithm which acts directly on the image as detected by the coils of the magnetic resonance apparatus and which subsequent encoding steps can be skipped and hence an acceleration of the signal acquisition for imaging by a factor of from two to three can be obtained. Crucial for the SENSE method is the knowledge of the sensitivity of the coils which are arranged in so called sensitivity maps. In order to accelerate this method there are proposals to use raw sensitivity maps which can be obtained through division by either the “sum-of-squares” of the single coil references or by an optional body coil reference (see e.g. K Pruessmann et. al. in Proc. ISMRM, 1998, abstracts pp. 579, 799, 803 and 2087). In fact the SENSE method allows for a decrease in scan time by deliberately undersampling k-space, i.e. deliberately selecting a Field-of-View (FOV) that is smaller than the object to be acquired. From this undersampling fold-over artefacts are obtained which can be resolved or unfolded by the use of the knowledge of a set of distinct coils having different coil sensitivity patterns. The undersampling can be in either one of both phase-encoding directions.

According to the first mentioned method the phase navigator signals are measured per single coil element of an array of multiple receiver coils, i.e. with the same coil element as was used for imaging. Also phase correction is applied per single coil. This way of correction can have two unwanted consequences:

1. if one needs the phase relation between the coils or synergy channels, as necessary for instance with the SENSE method, the corrected signals can be disturbed by a difference between the phase corrections per channel,

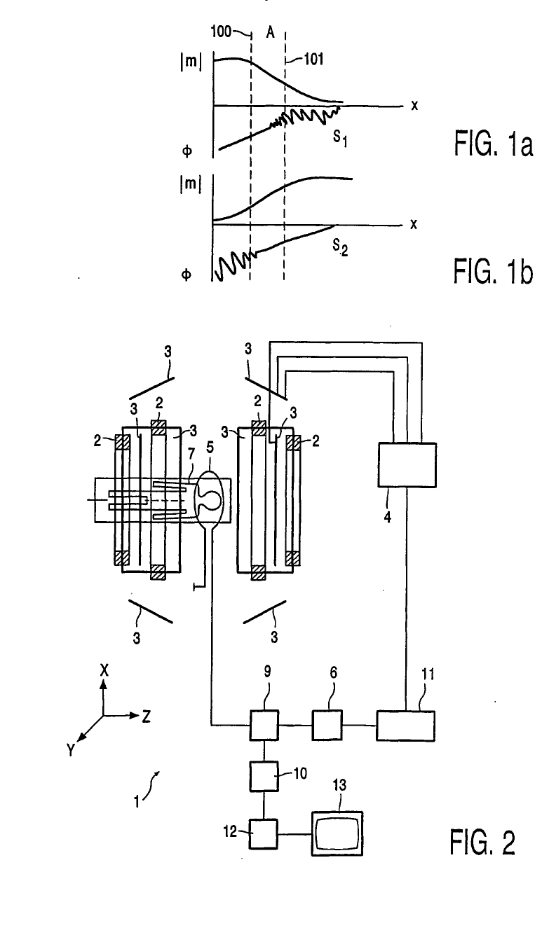

2. in regions where the coils or synergy channels measure a too low signal, the correction will be applied with high noise, which destroys phase encoding accuracy, resulting in many artefacts in the image region. In FIGS. 1a and 1b the modulus of the gradient signal m in x-direction and the related phase correction signal Φ is given for two synergy elements S₁ and S₂. The direction x of the measurement is running from element S₁ to S₂. In the region A between both dashed lines 100 and 101 the phase correction may cause problems because of a high noise level.

It is thus an object of the present invention to prevent aliasing in diffusion-weighted MR imaging.

This object of the invention is achieved by a method as defined in claim 1. The invention is further related to an apparatus as defined in claim 6 and to a computer program product as defined in claim 7.

These and other advantages of the invention are disclosed in the dependent claims and in the following description in which an exemplified embodiment of the invention is described with respect to the accompanying drawings. Therein, FIG. 2 shows an MR device which includes a first magnet system 2 for generating a steady magnetic field, and also means for generating additional magnetic fields having a gradient in the X, Y, Z directions, which means are known as gradient coils 3. The Z direction of the co-ordinate system shown corresponds to the direction of the steady magnetic field in the magnet system 2 by convention. The measuring co-ordinate system x, y, z to be used can be chosen independently of the X, Y, Z system shown in FIG. 2. The gradient coils or antennae are fed by a power supply unit 4. An RF transmitter coil 5 serves to generate RF magnetic fields and is connected to an RF transmitter and modulator 6. A receiver coil is used to receive the magnetic resonance signal generated by the RF field in the object 7 to be examined, for example a human or animal body. This coil may be the same coil as the RF transmitter coil 5 or an array of multiple receiver antennae (not shown). The coil 5 is a non phased-array receiver antenna, which is different from the array of multiple receiver antennae. Furthermore, the magnet system 2 encloses an examination space which is large enough to accommodate a part of the body 7 to be examined. The RF coil 5 is arranged around or on the part of the body 7 to be examined in this examination space. The RF transmitter coil 5 is connected to a signal amplifier and demodulation unit 10 via a transmission/reception circuit 9. The control unit 11 controls the RF transmitter and modulator 6 and the power supply unit 4 so as to generate special pulse sequences which contain RF pulses and gradients. The control unit 11 also controls detection of the MR signal(s), whose phase and amplitude obtained from the demodulation unit 10 are applied to a processing unit 12. The control unit 11 and the respective receiver coils 3 and 5 are equipped with control means to enable switching between their detection pathways on a sub-repetition time basis (i.e. typically less than 10 ms). These means comprise inter alia a current/voltage stabilization unit to ensure reliable phase behavior of the antennae, and one or more switches and analogue-to-digital converters in the signal path between coil and processing unit 12. The processing unit 12 processes the presented signal values so as to form an image by transformation. This image can be visualized, for example by means of a monitor 13.

The invention will be described hereinafter, by way of example, on the basis of versions of a method in which diffusion weighting is used in combination with a known echo planar imaging (EPI) pulse sequence so as to generate an MR signal. These EPI pulse sequences can be used to form an image by means of a two-dimensional or three-dimensional Fourier imaging technique. Another imaging technique for use of the present invention is SENSE as described in more detail in the above mentioned article of K. Pruesmann et. al.

The gist of the present invention is the use of a common (shared) correction vector for data of each separate coil or synergy channel. This common vector can be obtained from a data acquisition employing a different, volume encompassing coil or it can be derived as the weighted phase difference between a reference navigator acquisition and an actual navigator acquisition using the array of multiple receiver antennae. The weighting factor can either be the modulus of the reference navigator signal or can be the modulus of the not diffusion weighted signal at b=0.

Mathematically, both methods can be described as follows:

$$\begin{matrix}\text{Method 1:} \\ q ( x ) = {\sum }_{i} \frac{{n ( x )}_{R , i}^{*} \cdot {n ( x )}_{a , i}}{\vert {n ( x )}_{a , i} \vert}\end{matrix}$$

whereas n(x)_{R,i} is the reference navigator signal in the hybrid space (x, k_{y}) for coil i, and n(x)_{a,t} is the actual navigator signal in the hybrid space (x, k_{y}) with k_{y}=0 for coil i. In this case is

$$Δ = \frac{q ( x )}{\vert q ( x ) \vert}$$

the correction vector.

$$\begin{matrix}\text{Method 2:} \\ q ( x ) = {\sum }_{i} \frac{{n ( x )}_{R , i}^{*} \cdot {n ( x )}_{a , i}}{\vert {n ( x )}_{R , i} \vert \cdot \vert {n ( x )}_{a , i} \vert} \cdot \vert {n ( x )}_{b = 0 , i} \vert\end{matrix}$$

This means that the modulus of n(x)ᵢ at b=0 for each coil i is the weighting factor. Here also the correction vector is

$$Δ = \frac{q ( x )}{\vert q ( x ) \vert}$$

1. A magnetic resonance imaging method for forming an image of an object from a plurality of signals acquired by an array of multiple receiver antennae, wherein
spins are excitated in a part of the object,
MR signals are measured along a predetermined trajectory containing a plurality of lines in k-space by application of a read gradient and other gradients,
a navigator gradient is applied for the measurement of navigator MR signals, wherein phase corrections are determined from phases and moduli of the navigator MR signals so as to correct the measured MR signals and an image of the part of the object is determined from the corrected MR signals,
characterized in that a common correction vector is used for correction of data from all receiver antennae of the array.

2. A method as claimed in claim 1, characterized in that the common correction vector is determined from the weighted phase difference between a reference navigator signal for each antenna and the actual navigator MR signal of said antenna.

3. A method as claimed in claim 1, characterized in that the common correction vector is acquired from a non phased-array receiver antenna, different from the array of multiple receiver antennae being used for MR image data acquisition.

4. A method as claimed in claim 2, characterized in that the weighting factor is the modulus of the reference navigator signals.

5. A method as claimed in claim 2, characterized in that an additional gradient is applied to generate diffusion weighting and that the weighting factor is the modulus of the navigator signal without diffusion weighting.

6. A magnetic resonance imaging apparatus for obtaining an MR image from a plurality of signals comprising:
means for excitation of spins in apart of the object,
means for measuring MR signals along a predetermined trajectory containing a plurality of lines in k-space by application of a read gradient and other gradients,
means for applying a navigator gradient for the measurement of navigator MR signals and an additional gradient is applied in order to achieve diffusion sensitivity of the MR signal, wherein phase corrections are determined from phases and moduli of the navigator MR signals so as to correct the measured MR signals and an image of the part of the object is determined from the corrected MR signals, and
means for applying a common correction vector, which is used for correction of data from all receiver antennae of the array.

7. An apparatus as claimed in claim 6, characterized in that means are provided for determining the common correction vector from the weighted phase difference between a reference navigator for each antenna and the actual navigator MR signal of said antenna.

8. An apparatus as claimed in claim 6, characterized in that means are provided for acquiring the common correction vector from a non phased-array receiver antenna, different from the array of multiple receiver antennae being used for MR image data acquisition, further containing means for reliably switching between acquisition with the non phased-array antenna and acquisition with the array of multiple receiver antennae on a sub-repetition time basis.

9. A computer program product stored on a computer usable medium for forming an image by means of the magnetic resonance method, comprising a computer readable program means for causing the computer to control the execution of:
excitation of spins in a part of the object,
measuring of MR signals along a predetermined trajectory containing a plurality of lines in k-space by application of a read gradient and other gradients,
applying a navigator gradient for the measurement of navigator MR signals, wherein phase corrections are determined from phases and moduli of the navigator MR signals so as to correct the measured MR signals and an image of the part of the object is determined from the corrected MR signals,
using a common correction vector for correction of data from all receiver antennae of the array.

10. A computer program product as claimed in claim 9, wherein in addition to the navigator gradient a reference navigator gradient is applied in order to achieve diffusion sensitivity of the MR signal, and the corrected phase is determined from the weighted phase difference between the reference navigator signal for each antenna and the actual navigator MR signal of said antenna.
